(19) United States
(12) Patent Application Publication
(10) Pub. No.: US 2019/0374089 A1
(43) Pub. Date: Dec. 12, 2019

(54) FLEXIBLE TUBE INSERTION APPARATUS AND FLEXIBLE TUBE INSERTION METHOD

(71) Applicant: OLYMPUS CORPORATION, Tokyo (JP)

(72) Inventor: Shuji NAKAMURA, Akishima-shi (JP)

(73) Assignee: OLYMPUS CORPORATION, Tokyo (JP)

(21) Appl. No.: 16/449,518

(22) Filed: Jun. 24, 2019

Related U.S. Application Data
(63) Continuation of application No. PCT/JP2016/088942, filed on Dec. 27, 2016.

(51) Int. Cl. A61B 1/005; A61B 1/00; A61B 1/31

(52) U.S. Cl. CPC A61B 1/0051; A61B 1/00114; A61B 1/31; A61B 1/00078

(57) ABSTRACT

A The flexible tube insertion apparatus includes: a flexible tube section divided into one or more segments; one or more variable stiffness sections to change bending stiffness of the flexible tube section in a segment unit; a state detector to detect information on a bending state of the flexible tube section; a bend determination section to determine whether the flexible tube section bends based on the detected bending state; and a stiffness controller to control the bending stiffness of the flexible tube section in at least one segment unit by changing a bending stiffness value of the variable stiffness section based on information acquired from the bend determination section. After the bend determination section determines that a segment including the variable stiffness section bends, the stiffness controller controls the variable stiffness section so that a bending stiffness value of the flexible tube section in the segment becomes relatively high to a bending stiffness value of the flexible tube section at a proximal end side from the segment.

### CROSS-REFERENCE TO RELATED APPLICATIONS

This application is a Continuation Application of PCT Application No. PCT/JP2016/088942, filed Dec. 27, 2016, the entire contents of which are incorporated herein by reference.

### BACKGROUND OF THE INVENTION

### 1. Field of the Invention

The present invention relates to a flexible tube insertion apparatus comprising a flexible tube section to be inserted into an insertion target body and a flexible tube insertion method.

### 2. Description of the Related Art

In general, a sigmoid colon and a transverse colon in a large intestine are not fixed in an abdominal region, so as to easily move. When a flexible tube section of a flexible tube insertion apparatus (for example, an endoscope apparatus) is inserted into an intestine tract like this, the flexible tube section is bent along an intestine wall when the flexible tube section passes through a bent portion of the intestine tract, for example. If a user adds a force from a hand side and further pushes the flexible tube section therein at this time, the flexible tube section can bend in a different direction from a direction of transmission of the force in the intestine tract. Thereupon, a propulsive force at a distal end of the flexible tube section cannot be obtained, so that insertability is reduced.

In order to cope with the situation like this, attempts to improve insertability of an endoscope have been made. For example, in an endoscope disclosed in Jpn. Pat. Appln. KOKOKU publication No. 61-37931, an insertion section including a flexible tube section is divided into ranges in a longitudinal direction, and hardness of the flexible tube section is set so that degrees of flexibility in the respective ranges differ from one another.

Jpn. Pat. Appln. KOKAI Publication No. 6-70879 discloses an endoscope apparatus in which segments are set to an insertion section, and flexibility of the insertion section is controllable in each of the segments. In the endoscope apparatus, flexibilities of the respective segments are changed by using shape information of an endoscope, and a database storing a plurality of flexibility patterns based on past insertions.

Jpn. Pat. Appln. KOKAI Publication No. 2016-7434 discloses an endoscope apparatus in which an insertion section is divided into segments in a longitudinal direction, a bending shape of each of the segments is detected, and bending stiffness of each of the segments is changed in response to the detected bending shape.

### BRIEF SUMMARY OF THE INVENTION

An embodiment according to the present invention is a flexible tube insertion apparatus. The flexible tube insertion apparatus includes: a flexible tube section that is divided into one or more segments along an axial direction from a distal end side to a proximal end side, and is to be inserted into an insertion target body; at least one variable stiffness section that is disposed in the flexible tube section, and is configured to change bending stiffness of the flexible tube section in the segment unit; a state detector that is configured to detect a bending state of the flexible tube section; a bend determination section that is configured to determine whether or not the flexible tube section bends based on the detected bending state; and a stiffness controller that is configured to control the bending stiffness of the flexible tube section in the at least one segment unit by changing a bending stiffness value of the variable stiffness section based on information acquired from the bend determination section. After the bend determination section determines that a segment including the variable stiffness section bends, the stiffness controller controls the variable stiffness section so that a bending stiffness value of the flexible tube section in the segment becomes relatively high with respect to a bending stiffness value of the flexible tube section at a proximal end side from the segment.

Another embodiment according to the present invention is a flexible tube insertion method. The flexible tube includes a flexible tube section that is to be inserted into an insertion target body. The flexible tube section is divided into one or more segments along an axial direction from a distal end side to a proximal end side, and is provided with at least one variable stiffness section that changes bending stiffness of the flexible tube section in the segment unit. The flexible tube insertion method includes: detecting a bending state of the flexible tube section that is to be inserted into an insertion target body; determining whether or not a segment including the variable stiffness section bends based on the detected bending state; and controlling the variable stiffness section, after determining that the segment including the variable stiffness section bends, so that a bending stiffness value of the flexible tube section in the segment becomes relatively high with respect to a bending stiffness value of the flexible tube section at a proximal end side from the segment.

Advantages of the invention will be set forth in the description that follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a view schematically illustrating an example of an endoscope apparatus.

FIG. 2 is a view schematically illustrating an example of a flexible tube section of an endoscope in a first embodiment.

FIG. 3 is a block diagram illustrating an example of an endoscope apparatus in the first embodiment.

FIG. 4 is a view schematically illustrating an example of a variable stiffness section.

FIG. 5 is a diagram illustrating an example of a voltage-bending stiffness characteristic of the variable stiffness section.

FIG. 6 is a diagram of the flexible tube section modeled by using a stiff link model.

FIG. 7 is a diagram showing a concept of modeling the flexible tube section at a time of being inserted into an insertion target body by using the stiff link model.

FIG. 8 is a diagram illustrating an example of a flow of stiffness control in the first embodiment.

FIG. 9A is a view illustrating an example of a state of the flexible tube section at a time of insertion.

FIG. 9B is a view illustrating an example of a state of the flexible tube section at the time of insertion.

FIG. 10A is a view illustrating an example of a state of the flexible tube section at a time of insertion.

FIG. 10B is a view illustrating an example of a state of the flexible tube section at the time of insertion.

FIG. 10C is a view illustrating an example of a state of the flexible tube section at the time of insertion.

FIG. 11 is a diagram illustrating an example of stiffness control of respective variable stiffness sections at a certain time.

FIG. 12 is a view schematically illustrating an example of a flexible tube section of an endoscope in a second embodiment.

FIG. 13 is a block diagram illustrating an example of an endoscope apparatus in the second embodiment (an insertability determination section is here).

FIG. 14 is a diagram illustrating an example of a flow of stiffness control in the second embodiment.

### DETAILED DESCRIPTION OF THE INVENTION

Hereinafter, respective embodiments of the present invention will be described with reference to the drawings. Hereinafter, as an example of a flexible tube insertion apparatus of the present invention, an endoscope apparatus will be cited and explained.

### First Embodiment

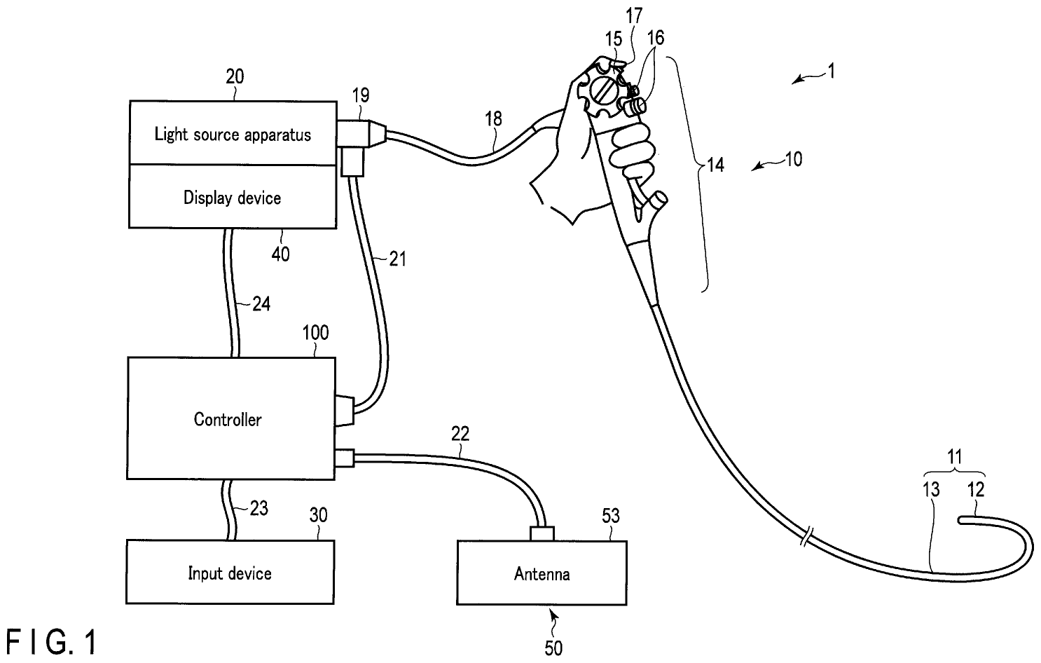

FIG. 1 is a view schematically illustrating an example of an endoscope apparatus 1. The endoscope apparatus 1 comprises an endoscope 10, a light source apparatus 20, an input device 30, a display device 40, an insertion shape detector 50, and a controller 100.

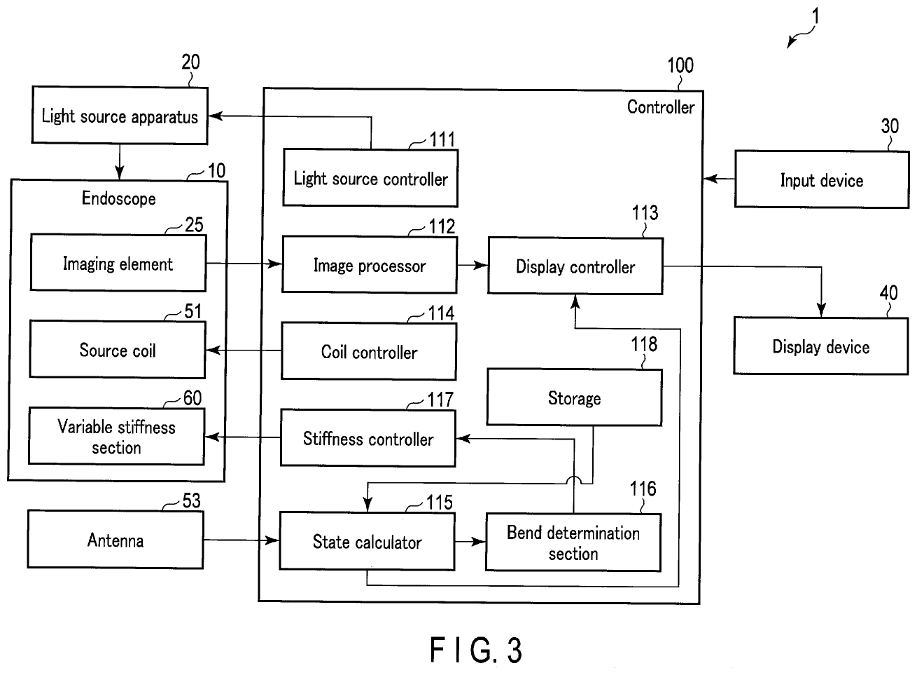

The endoscope 10 comprises a tubular insertion section 11 that is to be inserted into an insertion target body, and a control section 14 provided at a proximal end side of the insertion section 11. The insertion section 11 comprises a distal hard section 12, and a flexible tube section 13 provided at a proximal end side of the distal hard section 12. The distal hard section 12 comprises an illumination optical system and an observation optical system, which are not illustrated, an imaging element 25 illustrated in FIG. 3, and the like. The flexible tube section 13 is an elongated tubular section having flexibility. The control section 14 is provided with an angle knob 15 that is used for bend control of the endoscope 10, and one or more buttons 16 that are used for various controls including air-feeding, water-feeding, and suction controls. The flexible tube section 13 includes a bendable section at a distal end side, and the bendable section bends in an arbitrary direction by a user controlling the angle knob 15. Further, the control section 14 is provided with one or more switches 17 that are assigned with functions of stopping, recording, focus switching, and the like of an endoscope image by setting of the controller 100.

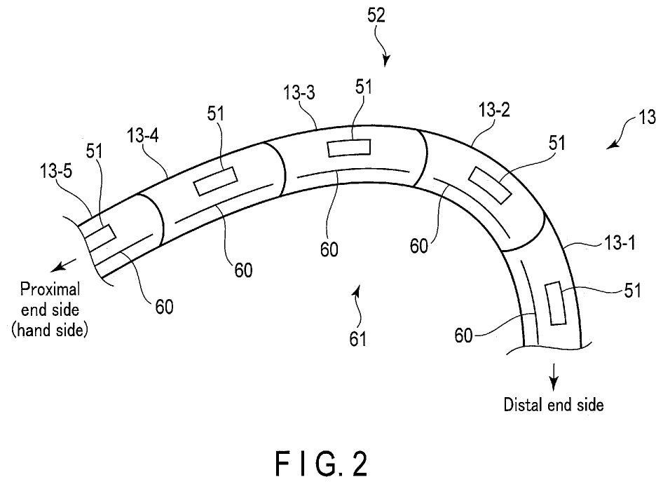

FIG. 2 is a view schematically illustrating an example of the flexible tube section 13 of the endoscope 10. In the flexible tube section 13, a source coil array 52 that includes source coils 51 for use in detection of a bending state of the flexible tube section 13 is disposed. The source coil 51 is configured by winding a leading wire around a magnetic body of a ferrite or Permalloy, for example. The source coil 51 is a magnetic field generating element that is configured to generate a magnetic field. An antenna 53 that is configured to detect the magnetic field generated by the source coil 51 is disposed around the insertion target body into which the insertion section 11 of the endoscope 10 is to be inserted, as illustrated in FIG. 1.

In the source coil array 52, the respective source coils 51 are disposed with spaces left from one another in a longitudinal direction (an axial direction) of the flexible tube section 13. For convenience, the flexible tube section 13 is assumed to comprise one or more segments (a virtual unit that equally divides the flexible tube section 13 in the longitudinal direction) that are taken along the axial direction of the flexible tube section 13. In other words, the flexible tube section 13 is assumed to be divided into one or more segments along the axial direction from the distal end side to the proximal end side. For example, FIG. 2 shows five segments 13-1, 13-2, 13-3, 13-4, and 13-5 that are arranged in series along the axial direction from the distal end side to the proximal end side, and the one source coil 51 is disposed in each of the segments. The source coils 51 that are provided in the respective segments are respectively disposed so that the antenna 53 and the controller 100 can detect information concerning bending states of the respective segments based on generated magnetic fields. In other words, the source coil array 52 (the respective source coils 51) is a state detector that is configured to detect a bending state of the flexible tube section 13 in segment units along a longitudinal direction of the insertion section 11. Note that disposition of the source coils 51 is not limited to this, but the source coils 51 may be disposed only in some of the segments.

In FIG. 2, the source coil 51 is incorporated in the flexible tube section 13 in advance, but the state detector is not limited to this. For example, a probe in which the source coil is incorporated may be inserted through an inside of a channel extending in the longitudinal direction in the insertion section 11.

Referring to FIG. 1 again, the light source apparatus 20 is connected to the endoscope 10 through a cable connector 19 at a distal end of a universal cable 18 extending from the control section 14. The universal cable 18 includes a light guide that is connected to the aforementioned illumination optical system, a transmission cable that is connected to the imaging element 25 and the like. The light source apparatus 20 includes ordinary light emitting elements such as laser diode (LD), and light emitting diode (LED). The light source apparatus 20 supplies illumination light that is emitted from an illumination window of the distal hard section 12 through the light guide.

FIG. 3 is a block diagram illustrating an example of the endoscope apparatus 1 in the first embodiment. The controller 100 is configured by devices including a CPU and the like. The controller 100 includes a light source controller 111, an image processor 112, a display controller 113, a coil controller 114, a state calculator 115, a bend determination section 116, a stiffness controller 117, and a storage 118. As illustrated in FIG. 1, the controller 100 is connected to the endoscope 10 and the light source apparatus 20 through the cable connector 19 and a cable 21. The controller 100 is also connected to the antenna 53 through a cable 22.

The light source controller 111 performs light control of the illumination light of the light source apparatus 20, and the like. The image processor 112 performs processing of converting an electric signal obtained by converting light from an object in the imaging element 25 of the endoscope 10 into a video signal. The display controller 113 controls an operation of the display device 40.

The coil controller 114 includes a coil output section that is configured to output voltages to be applied to the respective source coils 51 of the source coil array 52, and controls the voltages to be applied to the respective source coils 51 from the coil output section.

The state calculator 115 calculates position coordinates of the respective source coils 51 based on detection signals of the magnetic fields of the respective source coils 51, which are received by the antenna 53. In other words, the state calculator 115 calculates bending shape information of the flexible tube section 13, for example, radiuses R of curvature in the respective segments of the flexible tube section 13, based on information that is acquired from the respective source coils 51. Note that the state calculator 115 includes the receiver that is configured to receive detection signals from the antenna 53.

The bend determination section 116 determines the bending state of the flexible tube section 13 based on the bending shape information calculated by the state calculator 115. The stiffness controller 117 includes a variable stiffness output section that is configured to output voltages to be applied to a variable stiffness section 60, which will be described later, and controls a voltage to be applied to the variable stiffness section 60 from the variable stiffness output section.

The storage 118 stores a program including a calculation algorithm that is used in calculation of the bending state in the state calculator 115, and the like. The storage 118 may also store various kinds of information that are necessary in determination concerning the bending state of the flexible tube section 13 in the bend determination section 116. The storage 118 may be an external recording medium.

In the present embodiment, the respective source coils 51 of the source coil array 52, the antenna 53 that is disposed around the source coil array 52 (the respective source coils 51), the coil controller 114, and the state calculator 115 configure the insertion shape detector 50. The insertion shape detector 50 detects the magnetic fields generated by the respective source coils 51 of the source coil array 52 to observe the bending state of the insertion section 11 in order to support insertion of the insertion section 11 of the endoscope 10.

Note that the insertion shape detector 50 is not limited to the above. Any insertion shape detector that can detect the bending state of the flexible tube section 13 may be adopted, and the insertion shape detector can be configured by any one of sensing (electromagnetic sensor) using an electromagnetic wave, sensing (ultrasonic sensor) using an ultrasonic wave, sensing (optical fiber sensing) using loss of light, sensing (strain sensor) using a strain, and sensing using an X-ray absorbing material, or a combination of these sensors.

Next, the variable stiffness section 60 will be described. As illustrated in FIG. 2, the flexible tube section 13 is provided with a variable stiffness section array 61 including at least one variable stiffness section (variable stiffness actuator) 60. The respective variable stiffness sections 60 change bending stiffness (hardness) of the flexible tube section 13 in segment units with the segments where the respective variable stiffness sections 60 are provided as targets. The respective variable stiffness sections 60 can change bending stiffness of the segments where the variable stiffness sections 60 are provided, according to the respective segments, within a range from a predetermined minimal bending stiffness value to a maximal bending stiffness value.

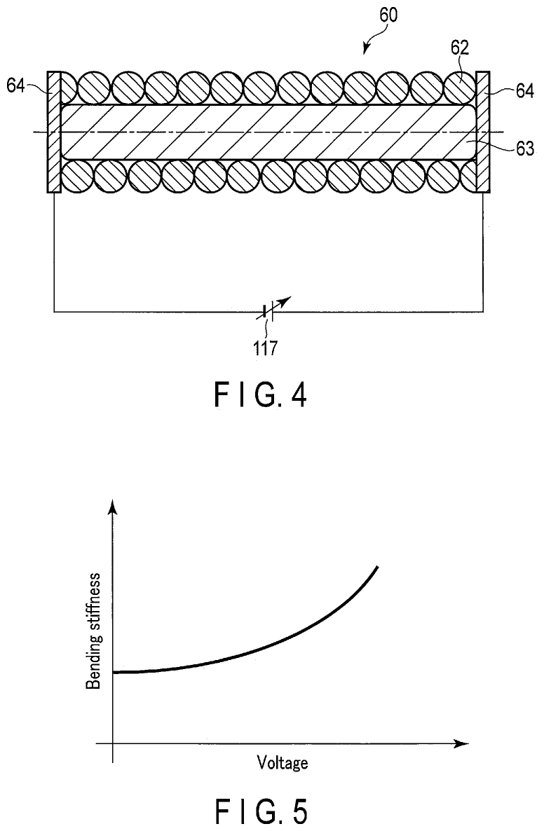

FIG. 4 is a view schematically illustrating an example of the variable stiffness section 60. The variable stiffness section 60 includes a coil pipe 62 formed of a metal wire, an electroactive polymer artificial muscle (Electroactive Polymer Artificial Muscle: EPAM) 63 that is sealed in the coil pipe 62, and electrodes 64 that are provided at both ends of the coil pipe 62. A voltage that is output from the stiffness controller 117 is applied to the EPAM 63 in the coil pipe 62 through the electrodes 64. The EPAM 63 is an actuator that is configured to extend and contract by applying a voltage, so as to change in hardness thereof. Each of the variable stiffness sections 60 is contained in the flexible tube section 13 so that a center axis of the coil pipe 62 coincides with or is parallel to a center axis of the flexible tube section 13. The EPAM 63 of each of the variable stiffness sections 60 has larger stiffness than stiffness of a material (for example, a fluororesin) forming the flexible tube section 13.

To the electrodes 64 (EPAM 63) of each of the variable stiffness sections 60, a voltage is applied by the stiffness controller 117 outputting the voltage from the variable stiffness output section thereof. When the voltage is applied to the EPAM 63, the EPAM 63 tries to extend a diameter of the EPAM 63 with the center axis of the coil pipe 62 as a center. However, a periphery of the EPAM 63 is surrounded by the coil pipe 62, so that extension of the diameter is restricted. Therefore, as illustrated in FIG. 5, in each of the variable stiffness sections 60, bending stiffness becomes higher, as a value of the voltage that is applied increases. In other words, by changing the hardness of the variable stiffness section 60, bending stiffness of the flexible tube section 13 in which the variable stiffness section 60 is contained also changes.

In this way, the endoscope apparatus 1 has a variable stiffness function of being able to change the bending stiffness of the flexible tube section 13 by the stiffness controller 117 applying the voltage to each of the variable stiffness sections 60 from the variable stiffness output section thereof. The stiffness controller 117 individually controls the voltage that is applied to each of the variable stiffness sections 60 from the variable stiffness output section thereof, and thereby bending stiffness (hardness) of each of the segments of the flexible tube section 13 is independently changed. In other words, it is possible to set a bending stiffness value that differs in each of the segments of the flexible tube section 13.

The input device 30 is an ordinary device for input such as a keyboard. The input device 30 is connected to the controller 100 through a cable 23. Various instructions and the like for operating the endoscope apparatus 1 are input to the input device 30. The input device 30 may be a control panel provided in the controller 100 or a touch panel displayed on a display screen.

The display device 40 is an ordinary monitor such as a liquid crystal display. The display device 40 is connected to the controller 100 through a cable 24. The display device 40 displays an endoscope observation image by a video signal that is transmitted from the image processor 112 of the controller 100. Further, the display device 40 displays a bending shape (a computer graphics image or text information) and the like of the flexible tube section 13 based on the position coordinates of the respective source coils 51 that are calculated in the state calculator 115 of the controller 100. The display device on which the endoscope observation image is displayed, and the display device on which the bending shape is displayed may be the same or separate.

Next, an operation of the endoscope apparatus 1 will be described. Hereinafter, as an example, it is assumed that the endoscope 10 is a large intestine endoscope, and an insertion target body is a large intestine. At a time of start of insertion, the flexible tube section 13 has a predetermined bending stiffness value (hardness), and the hardness is not the minimal bending stiffness value or the maximal bending stiffness value of the variable stiffness section 60. In other words, it is also possible to cause the respective segments of the flexible tube section 13 to be harder or softer after insertion than at the time of start of insertion.

The insertion section 11 of the endoscope 10 is inserted into a large intestine (from an anus to rectum, and colon) by the user. The insertion section 11 advances in an intestine tract while the insertion section 11 bends following a shape of the intestine tract. The endoscope 10 converts light from an object in the intestine tract into an electric signal by the imaging element 25 of the distal hard section 12. The electric signal is transmitted to the controller 100. The image processor 112 of the controller 100 acquires the electric signal to perform processing of converting the acquired electric signal into a video signal. The display controller 113 of the controller 100 causes the display device 40 to display an endoscope observation image based on the video signal.

During insertion, the coil controller 114 of the controller 100 applies a voltage to the respective source coils 51 from the coil output section. Thereby, the respective source coils 51 generate very weak magnetic fields around the respective source coils 51. In other words, information concerning positions of the respective source coils 51 is output from the respective source coils 51. The antenna 53 detects the magnetic fields generated by the source coils 51 to output detection signals to the state calculator 115.

The state calculator 115 receives the detection signal from the antenna 53 by the receiver to calculate the bending state, for example, a three-dimensional shape of the flexible tube section 13 based on the detection signal. Based on information on the calculated bending state, the display controller 113 generates a three-dimensional image corresponding to the information to cause the display device 40 to display the three-dimensional image. Further, the state calculator 115 calculates the state quantities indicating the bending states of the respective segments based on the calculated bending state of the flexible tube section 13.

The bend determination section 116 acquires the state quantities of the respective segments, which are calculated by the state calculator 115. The bend determination section 116 determines whether or not the respective segments bend based on the acquired state quantities and an arbitrary threshold of the state quantity input to the input device 30 by the user or a threshold of the state quantity that is acquired from the storage 118. Based on the determination, the stiffness controller 117 changes bending stiffness of the variable stiffness section 60.

In this way, in the endoscope apparatus 1, in response to the bending state of the flexible tube section 13 at the time of insertion, the stiffness controller 117 drives the variable stiffness sections 60 to change the bending stiffness of the flexible tube section 13.

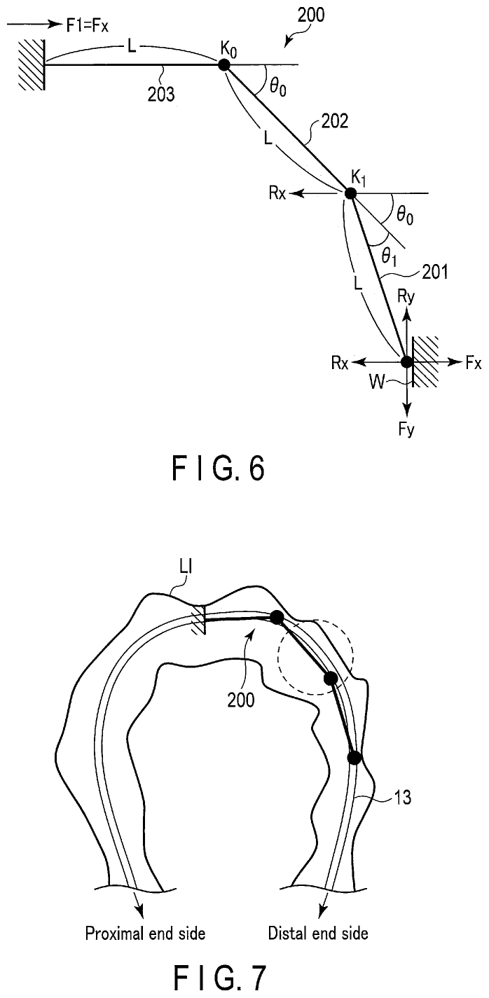

Next, a change of the bending stiffness of each of the segments of the flexible tube section 13 in the present embodiment will be theoretically explained by using FIG. 6 and FIG. 7.

FIG. 6 is a diagram of modeling the flexible tube section 13 of the endoscope 10 by using a stiff link model 200. The stiff link model 200 in which three stiff links 201, 202, and 203 are connected is considered. An entire length of each of the stiff links 201, 202, and 203 is L. Now, it is assumed that a force F1 is applied to a proximal end of the stiff link 203 at a hand side, and a distal end of the stiff link 201 at a distal end side is in a state of being collided with a wall W that imitates an intestine wall. Increasing a propulsive force Fy at the distal end of the stiff link 201 is considered in this state. Equations of torque balance in FIG. 6 are expressed as following equations (1) and (2).

T₀=K₀θ₀=Rx·L sin θ₀−Ry·L cos θ₀+T₁ equation (1)

T₁=K₁θ₁=Rx·L sin(θ₀+θ₁)−Ry·L cos(θ₀+θ₁) equation (2)

Here, T₀ and T₁ are respectively torques of rotating sections between the stiff links 203 and 202 and between the stiff links 202 and 201, K₀ and K₁ are rotational spring stiffness values (rotational spring constants), θ₀ and θ₁ are respectively angles of rotation shown in FIG. 6, Fx is a force that is given to the wall W by the stiff link 201, Rx is a reaction force of Fx(=F1), and Ry is a reaction force of the propulsive force Fy. When the reaction forces Rx and Ry are made subjects of equations (1) and (2), equations (3) and (4) as follows are obtained.

Rx={T₁ cos θ₀+(T₁−T₀)cos(θ₀+θ₁)}/L sin θ₁ equation (3)

Ry={T₁ sin θ₀+(T₁−T₀)sin(θ₀+θ₁)}/L sin θ₁ equation (4)

Here, from the law of action reaction, Fx=Rx=F1, and Fy=Ry are established. When T₀=K₀θ₀, and T₁=K₁θ₁ are substituted into equations (3) and (4), equation (5) is obtained.

Fy=Ry={K₁θ₁(sin θ₀+sin(θ₀+θ₁))−K₀θ₀·sin(θ₀+θ₁)}/L sin θ₁ equation (5)

From equation (5), it is found that in order to increase the propulsive force Fy at the distal end of the stiff link 201, K₁>K₀ is sufficient concerning rotational spring stiffness. When in the stiff link model 200, a bending stiffness value of the stiff link at a distal end side of the stiff link model 200 is larger than a bending stiffness value of the stiff link at a distal end side of the stiff link model 200, the propulsive force Fy increases. In the present embodiment, the above theory is applied to insertion of the flexible tube that passes through a bent portion.

FIG. 7 is a view showing a concept of modeling the flexible tube section 13 at the time of insertion by using the aforementioned stiff link model 200. Based on the aforementioned theory, the propulsive force at the distal end of the stiff link 201 increases, when the rotational spring stiffness value K₁ is caused to be larger than the rotational spring stiffness value K₀, that is, when a bending stiffness value of a segment at the distal end side of the flexible tube section 13 is caused to be larger than a bending stiffness value of a segment at the proximal end side. As a result, the flexible tube section 13 easily advances forward, so that insertability (ease of insertion) of the insertion section 11 is enhanced. For example, when a bending stiffness value of a segment of the flexible tube section 13 in a location shown by a circle of a broken line in FIG. 7 is larger than a bending stiffness value at the hand side, favorable insertion of the flexible tube section 13 is enabled.

From the above, in the present embodiment, the bending stiffness value at the distal end side of the flexible tube section 13 is caused to be relatively higher than the bending stiffness value at the proximal end side (hand side), when the flexible tube section 13 bends by a predetermined value or more. This increases the propulsive force at the distal end of the flexible tube section to enhance insertability.

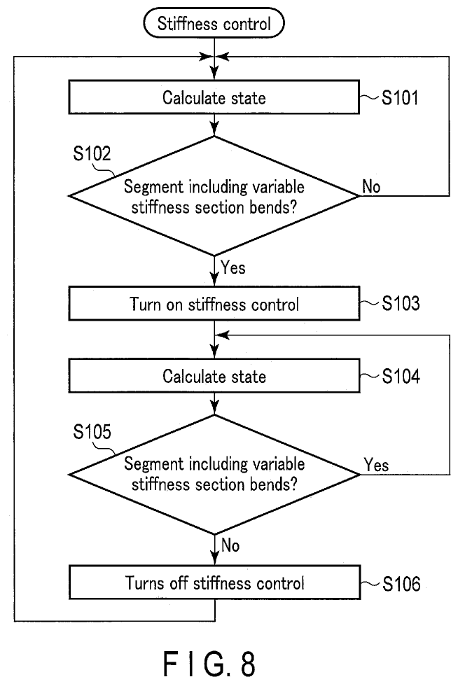

FIG. 8 is a diagram illustrating an example of a flow of stiffness control by the controller 100 in the first embodiment.

(In the Case of One Variable Stiffness Section)

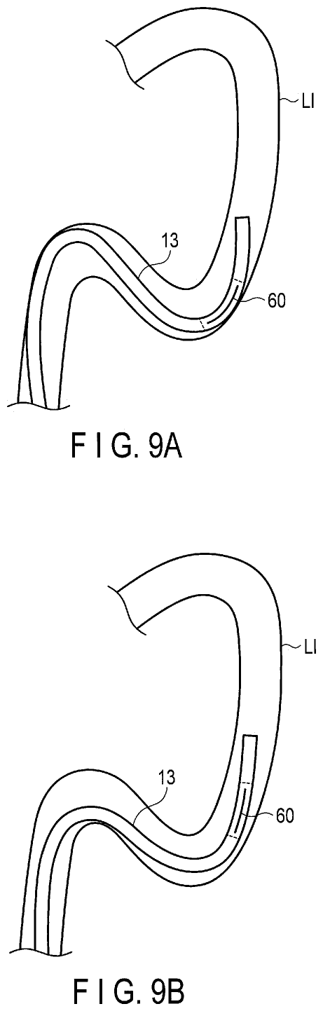

The flexible tube section 13 is assumed to include one segment and the one variable stiffness section 60 that is provided in the segment. FIG. 9A and FIG. 9B are schematic views illustrating an example of a state of the flexible tube section 13 in a case of the flexible tube section 13 including the one variable stiffness section 60.

In step S101, the state calculator 115 calculates the state quantity indicating the bending state of the segment of the flexible tube section 13. For example, the state calculator 115 calculates the radius R of curvature in the segment of the flexible tube section 13. The bend determination section 116 acquires the state quantity of the segment that is calculated by the state calculator 115. Further, the bend determination section 116 acquires a set value concerning the state quantity that is input to the input device 30 by a user, for example, a threshold of the radius of curvature. Alternatively, the bend determination section 116 may acquire the threshold value of the radius of curvature that is stored in the storage 118 in advance.

In step S102, the bend determination section 116 determines whether or not the segment including the variable stiffness section 60 bends. This can be determined based on whether or not the radius R of curvature, which is calculated in step S101, is a predetermined threshold that is a predetermined radius of curvature or less, for example. When the bend determination section 116 determines that the segment including the variable stiffness section 60 does not bend (No), a process returns to step S101. In other words, steps S101 and S102 are repeated until the bend determination section 116 determines that the segment including the variable stiffness section 60 bends. When the bend determination section 116 determines that the segment including the variable stiffness section 60 bends (Yes), the process proceeds to step S103.

For example, in the flexible tube section 13 illustrated in FIG. 9A, a segment that is provided with the variable stiffness section 60 bends more greatly than the predetermined radius of curvature in a bent portion of a large intestine, and is in a situation where the segment hits an intestine wall L1, and further insertion is difficult. In the situation like this, the bend determination section 116 determines that the segment including the variable stiffness section 60 bends equally to or more greatly than the predetermined threshold (Yes) in step S102, and the process proceeds to step S103.

In step S103, the stiffness controller 117 changes the bending stiffness of the variable stiffness section 60 of the segment that is determined as bending (stiffness control ON). The stiffness controller 117 controls the output of the voltage to the variable stiffness section 60 so that a bending stiffness value of the variable stiffness section 60 increases. As a result, the bending stiffness value of the variable stiffness section 60 increases, so that the segment provided with the variable stiffness section 60 becomes harder than other parts of the flexible tube section 13 than this segment.

In this manner, the stiffness controller 117 causes the bending stiffness value at the distal end side of the flexible tube section 13 to be relatively higher than the bending stiffness value at the hand side, when the bend determination section 116 determines that the segment including the variable stiffness section 60 bends. Thereby, the propulsive force at the distal end of the flexible tube section is enhanced.

After the bending stiffness is changed, the state calculator 115 calculates a state quantity indicating the bending state of each segment of the flexible tube section 13 as in step S101 (step S104). In step S105, the bend determination section 116 determines whether or not the segment including the variable stiffness section 60 bends as in step S102.

When the bend determination section 116 determines that the segment including the variable stiffness section 60 bends (Yes), the process returns to step S104. In other words, steps S104 and 5105 are repeated until the bend determination section 116 determines that the segment including the variable stiffness section 60 does not bend. When the bend determination section 116 determines that the segment including the variable stiffness section 60 does not bend (No), the process proceeds to step S106.

For example, it is assumed that the bending stiffness value at the distal end side is caused to be relatively higher than the bending stiffness value at the hand side, so that the flexible tube section 13 obtains a propulsive force at the distal end and advances, so as to be in a state illustrated in FIG. 9B. In the flexible tube section 13 illustrated in FIG. 9B, the segment that is provided with the variable stiffness section 60 does not bend more greatly than the predetermined radius of curvature, and is in a situation of being smoothly insertable. In the situation like this, the bend determination section 116 determines that the segment including the variable stiffness section 60 does not bend (No) in step S105, and the process proceeds to step S106.

In step S106, the stiffness controller 117 changes the bending stiffness of the variable stiffness section 60 of the segment that is determined as not bending (stiffness control OFF). The stiffness controller 117 changes the output of the voltage to the variable stiffness section 60 so that the bending stiffness value of the variable stiffness section 60 returns to an original bending stiffness value, for example. Thereby, the bending stiffness value of the variable stiffness section 60 returns to the original bending stiffness value, and the segment that is provided with the variable stiffness section 60 returns to a same hardness as the other parts of the flexible tube section 13 than this segment.

After step S106, the process returns to step S101, and stiffness control by the controller 100 is continued. The endoscope apparatus 1 always detects a bending state of the segment including the variable stiffness section 60 during use, and properly controls the bending stiffness value of the variable stiffness section 60 based on the detected bending state.

In the present embodiment, after the bend determination section 116 determines that the segment including the variable stiffness section 60 of the flexible tube section 13 bends by a predetermined value or more in the insertion target body, the controller 100 controls the voltage to be applied to the variable stiffness section 60 from the variable stiffness output section of the stiffness controller 117 so that the bending stiffness value of the variable stiffness section 60 included in the segment becomes relatively high with respect to the bending stiffness value of the flexible tube section 13 at the proximal end side (hand side) from the segment. According to the present embodiment, the stiffness controller 117 controls the bending stiffness value of the variable stiffness section 60 so that the flexible tube section 13 at the distal end side is relatively harder than at the hand side, so that the propulsive force at the distal end of the flexible tube section can be increased. As a result, the flexible tube insertion apparatus that enables smooth advance of the insertion section 11 in the insertion target body can be provided.

For example, if the insertion target body is a large intestine, favorable insertion of the insertion section 11 is enabled while extension of the intestine tract that causes pain in a patient is suppressed. Accordingly, the flexible tube insertion apparatus that is safer to patients can be provided. Further, as a result of insertability being enhanced, efficiency of endoscopy is also enhanced.

Further, the endoscope apparatus 1 has the insertion shape detector 50. Therefore, the controller 100 can perform stiffness control of the flexible tube section 13 while always acquiring information on the bending state of the flexible tube section 13 from the insertion shape detector 50. Accordingly, while following intestine tract shape that changes complicatedly, of, for example, a sigmoid colon, a transverse colon, or the like, which can easily move in the abdominal region, with the insertion shape detector 50, the controller 100 can properly change the bending stiffness value of the flexible tube section 13 in accordance with the movement. Therefore, the flexible tube insertion apparatus with enhanced insertability can be provided.

(In the Case of Variable Stiffness Sections)

Even when the flexible tube section 13 includes segments and variable stiffness sections 60 provided in the segments, the controller 100 also performs stiffness control according to the flow of steps S101 to S106 illustrated in FIG. 8. In other words, even when a number of variable stiffness sections 60 is two or more, the controller 100 controls the bending stiffness value at the distal end side of the flexible tube section 13 to be relatively higher than the bending stiffness value at the hand side with the stiffness controller 117 after determination of the bending state by the bend determination section 116.

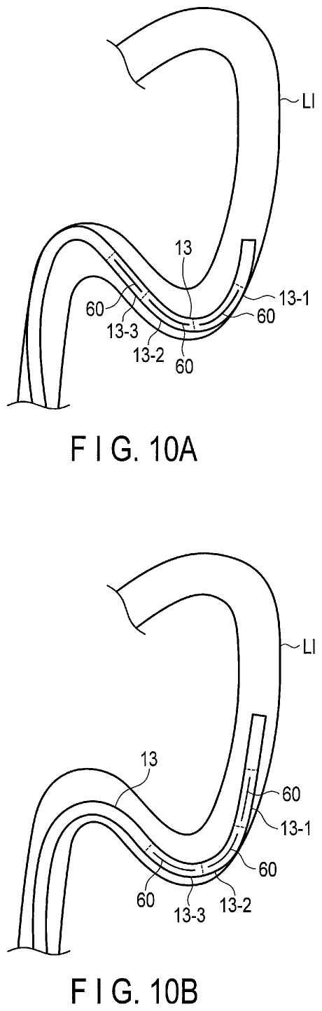

As an example, it is assumed that the flexible tube section 13 includes three segments 13-1, 13-2, and 13-3 in order from the distal end side thereof and three variable stiffness sections 60 that are provided in the segments. FIGS. 10A, 10B, and 10C are views illustrating an example of a state of the flexible tube section 13 in the case of the flexible tube section 13 including the three variable stiffness sections 60.

For example, in the flexible tube section 13 illustrated in FIG. 10A, the segment 13-1 bends more greatly than a predetermined radius of curvature in a bent portion of a large intestine, and is in a situation where the segment 13-1 hits an intestine wall L1 in the bent portion of the large intestine, and further insertion is difficult. In this situation, the stiffness controller 117 causes a bending stiffness value of the variable stiffness section 60 of the segment 13-1 to be higher than bending stiffness values of the variable stiffness sections 60 of the segments 13-2 and 13-3 at the hand side from the segment 13-1. As a result, the distal end of the flexible tube section 13 obtains a propulsive force and easily advances, and is soon brought into a state illustrated in FIG. 10B.

Next, in the flexible tube section 13 illustrated in FIG. 10B, the segment 13-2 bends more greatly than the predetermined radius of curvature in the bent portion of the large intestine, and is in a situation where the segment 13-2 hits the intestine wall L1 in the bent portion of the large intestine, and further insertion is difficult. In this situation, the stiffness controller 117 causes a bending stiffness value of the variable stiffness section 60 of the segment 13-2 to be higher than a bending stiffness value of the variable stiffness section 60 of the segment 13-3 at the hand side from the segment 13-2. As a result, the distal end of the flexible tube section 13 obtains a propulsive force and easily advances, and is soon brought into a state illustrated in FIG. 10C.

Further, in the flexible tube section 13 illustrated in FIG. 10C, the segment 13-3 bends more greatly than the predetermined radius of curvature in the bent portion of the large intestine, and is in a situation where the segment 13-3 hits the intestine wall L1 in the bent portion of the large intestine, and further insertion is difficult. In this situation, the stiffness controller 117 causes a bending stiffness value of the variable stiffness section 60 of the segment 13-3 to be higher than a bending stiffness value of a portion at the hand side from the segment 13-3. As a result, the distal end of the flexible tube section 13 obtains a propulsive force and easily advances.

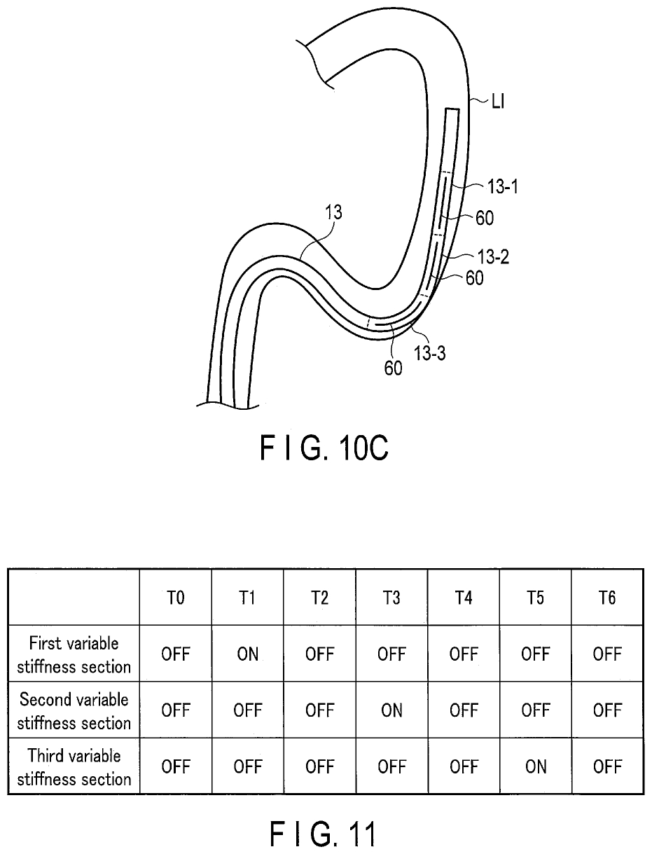

FIG. 11 is a diagram illustrating an example of stiffness control of the respective variable stiffness sections 60 in the case of the flexible tube section 13 including variable stiffness sections 60. In FIG. 11, the variable stiffness section 60 of the segment 13-1 is referred to as a first variable stiffness section, the variable stiffness section 60 of the segment 13-2 is referred to as a second variable stiffness section, and the variable stiffness section 60 of the segment 13-3 is referred to as a third variable stiffness section. At an initial time T0, the stiffness controller 117 of the controller 100 turns off the stiffness control of all the variable stiffness sections 60. Thereafter, the stiffness controller 117 turns on the stiffness control of the first variable stiffness section at a time T1 in step S103, and turns off the stiffness control of the first variable stiffness section at a time T2 in step S106. Thereafter, the stiffness controller 117 turns on the stiffness control of the second variable stiffness section at a time T3 in another step S103, and turns off the stiffness control of the second variable stiffness section at a time T4 in another step S106. Thereafter, the stiffness controller 117 turns on the stiffness control of the third variable stiffness section at a time T5 in still another step S103, and turns off the stiffness control of the third variable stiffness section at a time T6 in still another step S106.

For example, the flexible tube section 13 of the endoscope 10 at the time T1 is as illustrated in FIG. 10A. At the time T1, the stiffness controller 117 changes the bending stiffness of the first variable stiffness section 60 of the segment 13-1, which bends more greatly than the predetermined radius of curvature (stiffness control ON), but does not change the bending stiffnesses of the second variable stiffness section 60 of the segment 13-2 and the third variable stiffness section 60 of the segment 13-3, which do not bend more greatly than the predetermined radius of curvature (stiffness control OFF). The bending stiffness value of the first variable stiffness section 60 of the segment 13-1 is higher than the bending stiffness values of the second variable stiffness section 60 of the segment 13-2 and the third variable stiffness section 60 of the segment 13-3, which are at the hand side from the segment 13-1.

For example, the flexible tube section 13 of the endoscope 10 at the time T3 is as illustrated in FIG. 10B. At the time T3, the stiffness controller 117 changes the bending stiffness of the second variable stiffness section 60 of the segment 13-2, which bends more greatly than the predetermined radius of curvature (stiffness control ON), but does not change the bending stiffnesses of the first variable stiffness section 60 of the segment 13-1 and the third variable stiffness section 60 of the segment 13-3, which do not bend more greatly than the predetermined radius of curvature (stiffness control OFF). The bending stiffness value of the second variable stiffness section 60 of the segment 13-2 is higher than the bending stiffness value of the third variable stiffness section 60 of the segment 13-3 at the hand side from the segment 13-2.

For example, the flexible tube section 13 of the endoscope 10 at the time T5 is as illustrated in FIG. 10C. At the time T5, the stiffness controller 117 changes the bending stiffness of the third variable stiffness section 60 of the segment 13-3, which bends more greatly than the predetermined radius of curvature (stiffness control ON), but does not change the bending stiffnesses of the first variable stiffness section 60 of the segment 13-1 and the second variable stiffness section 60 of the segment 13-2, which do not bend more greatly than the predetermined radius of curvature (stiffness control OFF). The bending stiffness value of the third variable stiffness section 60 of the segment 13-3 is higher than the bending stiffness value of the flexible tube section 13 at the hand side from the segment 13-3.

In this way, when the stiffness controller 117 pays attention to the variable stiffness section 60 that is determined as bending by the bend determination section 116 and is intended to change the bending stiffness value, the stiffness controller 117 causes the bending stiffness value of the variable stiffness section 60 to be relatively higher than the bending stiffness values of the variable stiffness sections 60 at the hand side from that variable stiffness section 60 or the bending stiffness value of the flexible tube section 13 at the hand side from that variable stiffness section 60. By the control like this, the propulsive force at the distal end of the flexible tube section is enhanced. Therefore, the flexible tube insertion apparatus capable of smooth advance of the insertion section 11 in the insertion target body can be provided.

Further, paying attention to the time, when the number of variable stiffness sections 60 is two or more, the stiffness controller 117 controls the bending stiffness value of the variable stiffness section 60 of the segment of the flexible tube section 13 that is located at the distal end side earlier than, that is, before the bending stiffness value of the variable stiffness section 60 of the segment of the flexible tube section 13 that is located at the proximal end side. For example, as illustrated in FIG. 11, the stiffness controller 117 controls the bending stiffness values of the variable stiffness sections 60, which are provided in the respective segments, to be higher than the bending stiffness value of the variable stiffness section 60 that is provided in the segment at the proximal end side, in order from the distal end side. According to the control like this, the propulsive force at the distal end of the flexible tube section is increased, and insertability becomes favorable.

Even when the variable stiffness sections 60 are provided in the flexible tube section 13 in this way, the controller 100 controls the bending stiffness values of the respective variable stiffness sections 60 so that the force that pushes in the flexible tube section 13 is easily transmitted to the distal end of the flexible tube section from the hand side. Thereby, even if the insertion target body is an intestine tract in a complicated shape having bent portions, insertability can be enhanced.

Note that in the above explanation, after the bend determination section 116 determines that the segment including the variable stiffness section 60 bends, the stiffness controller 117 controls the bending stiffness value of the variable stiffness section 60 included in the segment to be relatively high with respect to the bending stiffness value of the flexible tube section 13 at the proximal end side from that segment. However, when attention is paid to a segment that does not bend, after the bend determination section 116 determines that the segment including the variable stiffness section 60 does not bend, the stiffness controller 117 may control the bending stiffness value of the variable stiffness section 60 included in the segment to be relatively low with respect to the bending stiffness value of the flexible tube section 13 at the distal end side from the segment. By the control like this, the bending stiffness value at the distal end side of the flexible tube section 13 also becomes relatively higher than the bending stiffness value at the proximal end side (hand side), so that the propulsive force of the distal end of the flexible tube section is enhanced, which contributes to enhancement of insertability.

Further, although the radius of curvature is cited as the state quantities of the respective segments that are calculated by the state calculator 115, a state quantity other than the radius of curvature may be used, such as bending angles or bending quantities in the respective segments. The bend determination section 116 may determine whether or not the flexible tube section 13 bends based on the state quantities like this that are acquired from the state calculator 115.

### Second Embodiment

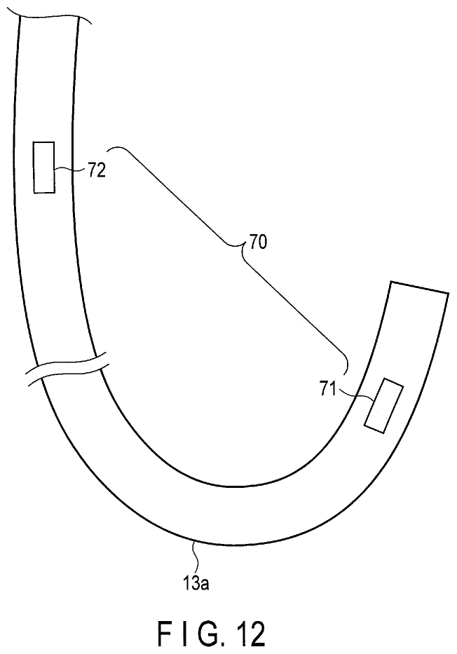

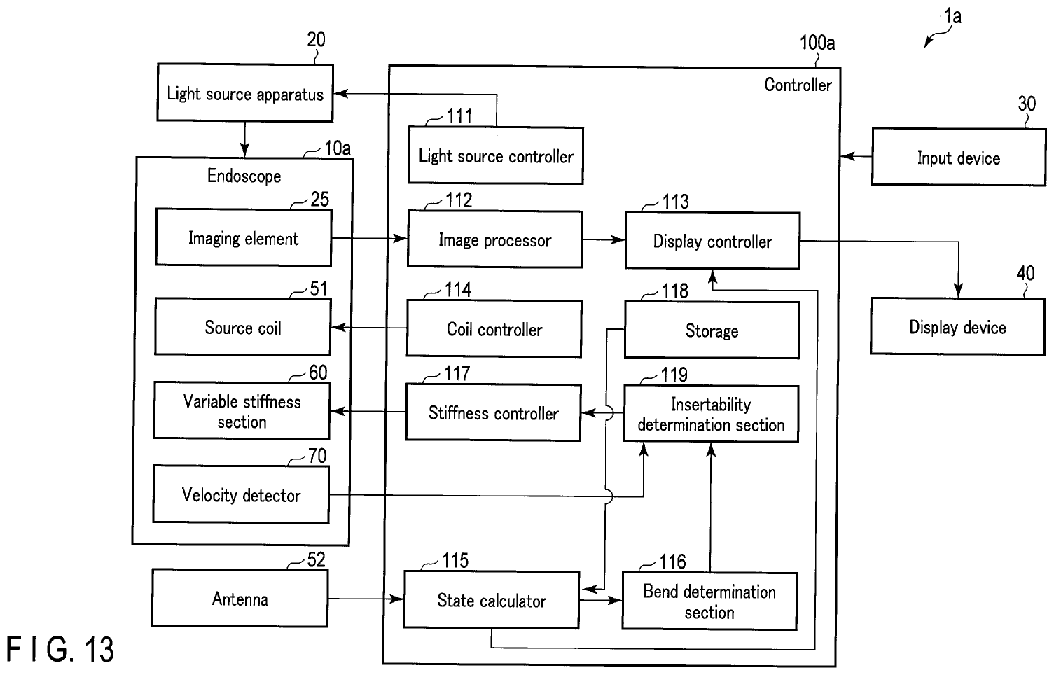

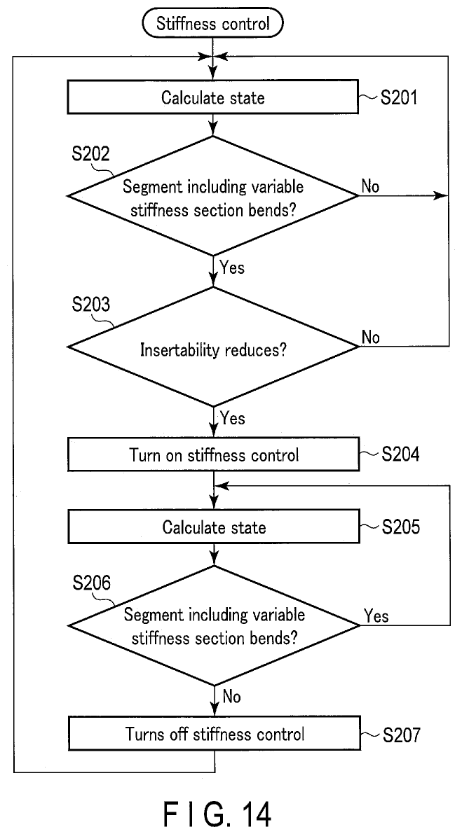

A second embodiment of the present invention will be described with reference to FIG. 12, FIG. 13, and FIG. 14. Hereinafter, explanation of the same components and operations as those in the first embodiment will be omitted, and a difference from the first embodiment will be mainly described. In the present embodiment, determination of reduction in insertability by an insertability determination section 119 is performed, in addition to determination of the bending state by the bend determination section 116.

FIG. 12 is a view schematically illustrating an example of a flexible tube section 13a of an endoscope 10a in the second embodiment. A velocity detector 70 is disposed in the flexible tube section 13a. As the velocity detector 70, for example, a first velocity sensor 71 is disposed at a distal end side of the flexible tube section 13a, and a second velocity sensor 72 is disposed at the hand side of the flexible tube section 13a, respectively. The velocity detector 70 detects a velocity at a location of the flexible tube section 13a where the velocity detector 70 is disposed. The velocity detector 70 may be an ordinary speed detector such as an acceleration sensor that is configured to detect a rate of velocity change to a time. The velocity detector 70 is contained in the flexible tube section 13a, for example, and is connected to a controller 100a.

FIG. 13 is a block diagram illustrating an example of an endoscope apparatus 1a in the second embodiment. The controller 100a includes the insertability determination section 119, in addition to the light source controller 111, the image processor 112, the display controller 113, the coil controller 114, the state calculator 115, the bend determination section 116, the stiffness controller 117, and the storage 118 as in the first embodiment. The insertability determination section 119 determines reduction in insertability of the flexible tube section 13a based on velocity information that is acquired from the velocity detector 70.

FIG. 14 is a diagram illustrating an example of a flow of stiffness control by the controller 100a in the second embodiment.

In step S201, the state calculator 115 calculates a state quantity indicating bending states of respective segments of the flexible tube section 13a. For example, the state calculator 115 calculates radiuses R of curvature in the respective segments of the flexible tube section 13a. The bend determination section 116 acquires the state quantities of the respective segments that are calculated by the state calculator 115. Further, the bend determination section 116 acquires a set value concerning a state quantity that is input to the input device 30 by a user, for example, a threshold of a radius of curvature. Alternatively, the bend determination section 116 may acquire a threshold of a radius of curvature that is stored in the storage 118 in advance.

In step S202, the bend determination section 116 determines whether or not a segment including a variable stiffness section 60 bends. When the bend determination section 116 determines that the segment including the variable stiffness section 60 does not bend (No), a process returns to step S201. In other words, steps S201 and S202 are repeated until the bend determination section 116 determines that the segment including the variable stiffness section 60 bends. When the bend determination section 116 determines that the segment including the variable stiffness section 60 bends (Yes), the process proceeds to step S203.

Unlike the first embodiment, in the second embodiment, the stiffness controller 117 does not perform stiffness control immediately, even when the bend determination section 116 determines that the segment including the variable stiffness section 60 bends in step S202. In step S203, the insertability determination section 119 determines whether or not insertability of the flexible tube section 13a is reduced. For example, the insertability determination section 119 acquires velocity information detected by the first velocity sensor 71 and the second velocity sensor 72 to determine that insertability is reduced when an insertion velocity by the first velocity sensor 71 is lower than an insertion velocity by the second velocity sensor 72. In other words, when an insertion velocity at a distal end side of the flexible tube section 13a is lower than an insertion velocity at the hand side, the insertability determination section 119 determines that insertability is reduced. When the insertion velocity at the distal end side is lower than the insertion velocity at the hand side, it is conceivable that the flexible tube section 13a at the distal end side does not advance even if the user pushes in the insertion section 11 from the hand side of the flexible tube section 13a, and is in a situation where further insertion is difficult.

When the insertability determination section 119 determines that insertability of the flexible tube section 13a is not reduced (No), the process returns to step S201. In other words, steps S201, S202, and S203 are repeated, until the bend determination section 116 determines that the segment including the variable stiffness section 60 bends, and the insertability determination section 119 determines that insertability of the flexible tube section 13a is reduced. In the present embodiment, even if the bend determination section 116 determines that the segment including the variable stiffness section 60 bends in step S202, the bending stiffness value of the variable stiffness section 60 is not changed unless reduction in insertability is confirmed.

When the insertability determination section 119 determines that insertability of the flexible tube section 13a is reduced (Yes) in step S203, the process proceeds to step S204. In other words, the process proceeds to step S204, when the bend determination section 116 determines that the segment including the variable stiffness section 60 bends, and the insertability determination section 119 determines that insertability of the flexible tube section 13a is reduced.

In step S204, the stiffness controller 117 changes the bending stiffness of the variable stiffness section 60 of the segment that is determined as bending in step S202 (stiffness control ON). The stiffness controller 117 controls the output of the voltage to the variable stiffness section 60 so that the bending stiffness value of the variable stiffness section 60 increases. As a result, the bending stiffness value of the variable stiffness section 60 increases, so that the segment that is provided with the variable stiffness section 60 becomes harder than the other segments or other parts of the flexible tube section 13a than this segment.

After the bending stiffness is changed, the state calculator 115 calculates the state quantities indicating the bending states of the respective segments of the flexible tube section 13a as in step S201 (step S205). Next, in step S206, the bend determination section 116 determines whether or not the segment including the variable stiffness section 60 bends in the same way as in step S202.

When the bend determination section 116 determines that the segment including the variable stiffness section 60 bends (Yes), the process returns to step S205. In other words, steps S205 and S206 are repeated until the bend determination section 116 determines that the segment including the variable stiffness section 60 does not bend. When the bend determination section 116 determines that the segment including the variable stiffness section 60 does not bend (No), the process proceeds to step S207.

In step S207, the stiffness controller 117 changes the bending stiffness of the variable stiffness section 60 of the segment that is determined as not bending (stiffness control OFF). The stiffness controller 117 changes the output of the voltage to the variable stiffness section 60 so that the bending stiffness value of the variable stiffness section 60 returns to an original bending stiffness value, for example. As a result, the bending stiffness value of the variable stiffness section 60 returns to the original bending stiffness value, so that a hardness of the segment provided with the variable stiffness section 60 returns to the same hardness as a hardness of the other segments or other parts of the flexible tube section 13a than this segment.

After step S207, the process returns to step S201, and stiffness control by the controller 100 is continued. In also the present embodiment, the endoscope apparatus 1a always detects the bending state of the segment including the variable stiffness section 60 during use, and properly controls the bending stiffness value of the variable stiffness section 60 based on the detected bending state.

In the present embodiment, the stiffness controller 117 controls the bending stiffness value of the flexible tube section located at the distal end side to be relatively higher than the bending stiffness value of the flexible tube section located at the hand side, after the bend determination section 116 determines that the segment including the variable stiffness section bends by a predetermined value or more, and the insertability determination section 119 determines that insertability of the flexible tube section 13a is reduced. According to the present embodiment, by using reduction in insertability based on a change of an insertion velocity of the flexible tube section 13a in addition to the bending state, in determination of the bending stiffness control of the variable stiffness section 60, it is more properly determined whether or not the flexible tube section 13a is in a state of requiring bending stiffness control in an insertion target body. The stiffness controller 117 changes the bending stiffness value after the controller 100 more properly determines the state of the flexible tube section 13a, so that the endoscope apparatus 1a that is adapted to a complicated bending shape in an intestine tract, and has more favorable insertability can be provided.

In also the second embodiment, after the bend determination section 116 determines that the segment including the variable stiffness section 60 does not bend, the stiffness controller 117 may control the bending stiffness value of the variable stiffness section 60 included in the segment to be relatively low with respect to the bending stiffness value of the flexible tube section 13a at the distal end side from the segment.

In the second embodiment, the insertability determination section 119 determines whether or not the insertability of the flexible tube section 13a is reduced based on the velocity information that is acquired from the velocity detector 70, but a user may perform determination of reduction in insertability. The user determines that insertability of the flexible tube section 13a is reduced when the user, for example, confirms that the flexible tube section 13a does not advance even if the user pushes in the flexible tube section 13a from the hand side, while watching the bending shape of the flexible tube section 13 that is displayed on the display device 40. When the user determines that the insertability of the flexible tube section 13a is reduced, the stiffness controller 117 is caused to change the bending stiffness of the variable stiffness section 60 of the bending segment.

While the respective embodiments of the present invention are described thus far, the present invention is not limited to the aforementioned embodiments, but various improvements and changes can be made within the range without departing from the gist of the present invention. For example, the flexible tube insertion apparatus is not limited to the endoscope apparatus, but it is obvious to a person skilled in the art that a wide variety of insertion apparatuses having insertion sections with flexibility (flexible tube sections) are included in the scope of the present invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A flexible tube insertion apparatus, comprising:
a flexible tube section that is divided into one or more segments along an axial direction from a distal end side to a proximal end side, and is to be inserted into an insertion target body;
at least one variable stiffness section that is disposed in the flexible tube section, and is configured to change bending stiffness of the flexible tube section in the segment unit;
a state detector that is configured to detect a bending state of the flexible tube section;
a bend determination section that is configured to determine whether or not the flexible tube section bends based on the detected bending state; and
a stiffness controller that is configured to control the bending stiffness of the flexible tube section in the at least one segment unit by changing a bending stiffness value of the variable stiffness section based on information acquired from the bend determination section,
after the bend determination section determines that a segment including the variable stiffness section bends, the stiffness controller controlling the variable stiffness section so that a bending stiffness value of the flexible tube section in the segment becomes relatively high with respect to a bending stiffness value of the flexible tube section at a proximal end side from the segment.

2. The flexible tube insertion apparatus according to claim 1, wherein in a case of including two or more of the variable stiffness sections, the stiffness controller controls a bending stiffness value of the variable stiffness section that is located at a distal end side earlier than a bending stiffness value of the variable stiffness section that is located at a proximal end side.

3. The flexible tube insertion apparatus according to claim 1, wherein after the bend determination section determines that the segment including the variable stiffness section does not bend, the stiffness controller controls a bending stiffness value of the variable stiffness section that is included in the segment to be relatively low with respect to a bending stiffness value of the flexible tube section at a distal end side from the segment.

4. The flexible tube insertion apparatus according to claim 1, further comprising:
a velocity detector that is configured to detect a velocity of the flexible tube section; and
an insertability determination section that is configured to determine whether or not insertability of the flexible tube section is reduced based on velocity information acquired from the velocity detector.

5. The flexible tube insertion apparatus according to claim 4, wherein after the bend determination section determines that the segment including the variable stiffness section bends, and after the insertability determination section determines that insertability of the flexible tube section is reduced, the stiffness controller controls the bending stiffness value of the variable stiffness section included in the segment to be relatively high with respect to the bending stiffness value of the flexible tube section at the proximal end side from the segment.

6. The flexible tube insertion apparatus according to claim 1, wherein the bend determination section determines that the flexible tube section bends when a radius of curvature of the flexible tube section is a predetermined threshold or less.

7. The flexible tube insertion apparatus according to claim 6, further comprising:
an input device,
wherein the threshold is arbitrarily set by a user with the input device.

8. The flexible tube insertion apparatus according to claim 4, wherein the insertability determination section determines that insertability is reduced when an insertion velocity at a distal end side of the flexible tube section that is detected by the velocity detector is lower than an insertion velocity at a proximal end side of the flexible tube section.

9. A flexible tube insertion method,
the flexible tube including a flexible tube section that is to be inserted into an insertion target body, the flexible tube section being divided into one or more segments along an axial direction from a distal end side to a proximal end side, and being provided with at least one variable stiffness section that changes bending stiffness of the flexible tube section in the segment unit, the flexible tube insertion method comprising:
detecting a bending state of the flexible tube section that is to be inserted into an insertion target body;
determining whether or not a segment including the variable stiffness section bends based on the detected bending state; and
controlling the variable stiffness section, after determining that the segment including the variable stiffness section bends, so that a bending stiffness value of the flexible tube section in the segment becomes relatively high with respect to a bending stiffness value of the flexible tube section at a proximal end side from the segment.

10. The flexible tube insertion method according to claim 9, wherein in a case of including two or more of the variable stiffness sections, the controlling the variable stiffness section comprises controlling a bending stiffness value of the variable stiffness section that is located at a distal end side earlier than a bending stiffness value of the variable stiffness section that is located at a proximal end side.

11. The flexible tube insertion method according to claim 9, wherein the controlling the variable stiffness section comprises controlling, after determining that the segment including the variable stiffness section does not bend, a bending stiffness value of the variable stiffness section included in the segment to be relatively low with respect to a bending stiffness value of the flexible tube section at a distal end side from the segment.

12. The flexible tube insertion method according to claim 9, further comprising:
detecting a velocity of the flexible tube section; and
determining whether or not insertability of the flexible tube section is reduced based on velocity information.

13. The flexible tube insertion method according to claim 12, wherein the controlling the variable stiffness section comprises controlling, after determining that the segment including the variable stiffness section bends and after determining that insertability of the flexible tube section is reduced, a bending stiffness value of the variable stiffness section included in the segment to be relatively high with respect to a bending stiffness value of the flexible tube section at a proximal end side from the segment.

14. The flexible tube insertion method according to claim 9, wherein the determining whether or not the segment bends comprises determining that the segment bends when a radius of curvature of the flexible tube section is a predetermined threshold or less.

15. The flexible tube insertion method according to claim 9, wherein the threshold is arbitrarily set by a user in an input device.

16. The flexible tube insertion method according to claim 12, wherein the determining whether or not insertability is reduced comprises determining that insertability is reduced when an insertion velocity at a distal end side of the flexible tube section that is detected is lower than an insertion velocity at a proximal end side of the flexible tube section.
